(19) United States
(12) Patent Application Publication
(10) Pub. No.: US 2019/0027060 A1
(43) Pub. Date: Jan. 24, 2019

(54) FOOD PROPOSING SYSTEM, FOOD PROPOSING METHOD AND FOOD PROPOSING PROGRAM

(71) Applicant: HITACHI, LTD., Tokyo (JP)

(72) Inventor: Tomoyuki ISHII, Tokyo (JP)

(73) Assignee: HITACHI, LTD., Tokyo (JP)

(21) Appl. No.: 15/777,136

(22) PCT Filed: Nov. 17, 2015
(86) PCT No.: PCT/JP2015/082195

§ 371 Date: Oct. 12, 2018

(51) Int. Cl. G09B 19/00; G16H 20/60

(52) U.S. Cl. CPC G09B 19/0092; G16H 20/60

(57) ABSTRACT

A food proposing system includes a storage section storing symptom and nutrient information and food and nutrient information, a symptom receiving and processing section that receives symptoms input by a user; an required nutrient determining section that searches the symptom and nutrient information on the input symptoms and then retrieves the effect degree of each nutrient for improving the symptoms; a food candidate determining section that computes a score of each food on the basis of the effect degree of the retrieved nutrient and the content thereof in the food included by the food and nutrient information and determines the foods by an arbitrarily preset number, the foods being to be proposed to the user, based on how large computed scores are; and a food candidate indicating and processing section that indicates the determined foods.

### TECHNICAL FIELD

The present invention relates to a food proposing system, a food proposing method, and a food proposing program.

### BACKGROUND

Lifestyle habits such as eating, exercising, and sleeping have significant effects on physical condition of a human. Among these lifestyle habits, eating is especially important. Since physical condition is reflected on the appearance of the human, eating is important also from a viewpoint of beauty. Further, eating is also enjoyment for the human, and it is possible for the human to maintain his/her health without being bored by properly devising the selection of food and menu. With this background, in recent years, there have been many technologies for supporting the selection of the food and menu widespread in the world.

A production and distribution system of Patent Document 1 stores a table in which physical condition, nutrient, food, menu, a production district of the food and a store are associated with each other. Further, the system of Patent Document 1 receives the input of a physical condition to display the nutrient, the food, the menu, the production district of the food and the store corresponding to the physical condition. A menu support system of Patent Document 2 stores a table in which food, nutrient, threshold value of the nutrient, and menu are associated with each other. Furthermore, the system of Patent Document 2 continuously monitors the inventory change of the foods, and when detecting the decrease in the inventory of a certain food, the system of Patent Document 2 considers that the nutrient in the food has been ingested to compare between the decrement of the ingestion and the threshold value. If the decrement exceeds the threshold value, the menu support system of Patent Document 2 displays the menu containing the nutrient.

### PRIOR ART DOCUMENTS

### Patent Documents

Patent Document 1: Japanese Published Unexamined Patent Application 2015-56022

Patent Document 2: Japanese Published Unexamined Patent Application 2013-250699

### SUMMARY OF INVENTION

### Technical Problem

In the table that is stored by the production and distribution system of Patent Document 1, the physical condition and the nutrient are in one-to-one correspondence. The food and the nutrient are also in one-to-one correspondence. Accordingly, only one food is determined as the food for improving a certain physical condition, depriving the user of choice to select the food. Furthermore, the more foods than user can select is proposed to the user who attempts simultaneously to improve the physical condition in many points. The proposed foods may contain unwanted (adverse) nutrients for the user. The production and distribution system of Patent Document 1 doesn't include the unit that records the food that the user has ingested. Generally, the production and distribution system of Patent Document 1 is suitable when a busy business person finds a restaurant or the like, however is not suitable for the person who has high health consciousness and cook for himself/herself.

The menu support system of Patent Document 2 doesn't ask the user his/her physical condition directly, but only asks the user what kinds of foods are decreased in the inventory by the user. Thus, the ingestions of foods that don't cause the decrease in the inventory (eating out, catering, etc.) are disregarded. Further, the menu support system of Patent Document 2 displays the menu rather than the food. In the first place, the indicated menu does not always match the preference of the user. Even if the indicated menu matches the preference of the user, the food unnecessary for improving the physical condition may be unavailable among the foods for cooking the menu. In this case, the priority is getting wrong in such a way that the user can't help giving up menu despite the foods required for improving the physical condition are available, which is a case of mistaking means for ends. The menu support system of Patent Document 2 is also not suitable for the user who stocks up on the necessary foods to be ingested on separate occasions as separate menus.

Accordingly, the object of present invention is to propose to the user who cook foods in their own way and timing to ingest the cooked foods, the foods really truly required for improving the symptoms as an appropriate number of candidates. In particular, the object of present invention is to adjust flexibly the number of the candidates by proposing an enough number of the candidates even when the input number of the symptoms is small, while by proposing a small number of the candidates that contribute simultaneously to the improvement degree of different kinds of the symptoms even when the input number of symptoms is large. Further, the object of present invention is to record appropriately the proposed foods and the foods selected by the user from among the proposed foods.

### Solution to Problems

The food proposing system of the present invention includes a food proposing system comprising: a storage section storing: symptom and nutrient information including symptoms, nutrients, an effect degree of each of the nutrients for improving each of the symptoms, for each combination of the symptoms and the nutrients and food and nutrient information including foods, the nutrients, a content of each of the nutrients contained in each of the foods, for each combination of the foods and the nutrients; a symptom receiving and processing section configured to receive symptoms input by a user; a required nutrient determining section configured to: search the symptom and nutrient information on the input symptoms and

retrieve the effect degree of each of the nutrients for improving the input symptoms; a food candidate determining section configured to: compute a score of each of the foods on the basis of the retrieved effect degree of the each of the nutrients and the content thereof in the each of the foods included by the food and nutrient information, and determine a food by an arbitrarily preset number, the food to be proposed to the user, based on how large computed scores are; and a food candidate indicating and processing section configured to indicate the determined food.

Other units will be described in the embodiment for implementing the invention.

### Advantageous Effects of Invention

According to the present invention, it is possible to propose the foods truly required for improving the symptoms to the user who attempts to cook in his/her own way and timing to ingest the cooked foods, as the appropriate number of the candidates.

### BRIEF DESCRIPTION OF DRAWINGS

FIG. 1 is a block diagram illustrating one example of a configuration of a food proposing system.

FIG. 2 is a diagram illustrating one example of an interview screen.

FIG. 3 is a diagram illustrating one example of a food proposing screen.

FIG. 4 is a graph illustrating one example of an achievement rate transition screen.

FIG. 5 is a diagram illustrating one example of a conversation screen.

FIG. 6 is a table illustrating one example of symptom reception information.

FIG. 7 is a table illustrating one example of food proposing information.

FIG. 8 is a table illustrating one example of symptom and nutrient information.

FIG. 9 is a table illustrating one example of the symptom and nutrient information.

FIG. 10 is a table illustrating one example of the symptom and nutrient information.

FIG. 11 is a table illustrating one example of the symptom and nutrient information.

FIG. 12 is a table illustrating one example of food group information.

FIG. 13 is a flowchart illustrating a processing procedure.

FIG. 14A and FIG. 14B are graphs illustrating the learning of effect degrees.

### DETAILED DESCRIPTION OF EMBODIMENTS

Hereinafter, the embodiments of the present invention (referred to as “present embodiment”) will be described in detail with reference to the drawings. In the present embodiment, the user of a food proposing system accesses the food proposing system from a portable terminal via a network to view an interview screen or the like (the detail of which will be described below).

### Terms

Symptom means a state in which the user worries about his/her own physical condition. For example, the representative examples of the symptoms are high blood pressure, high blood sugar and obese or the like, which are clearly related to a particular disease. The symptoms are not necessarily related to a particular disease. For example, the symptoms may also include the states of; disturbance of skin texture, dull skin, oily skin, and poor makeup application of skin, noticeable dark circles under the eyes, disheveled hair, a scurfy head, strong breath, languid, upset stomach, without appetite, stiff shoulders or the like. Further, the symptoms may include not only physical states but also psychological states. For example, the symptoms may include the states of being not concentrated and being frustrated or the like.

In addition, the symptoms may include lifestyle habits itself such as irregularity of dietary life and life pattern and sufferings in the middle of working on the improvement in the lifestyle habits. For example, the symptoms may include the states of; eating frequently midnight meal, waking up late, tobacco withdrawal symptom or the like. The present embodiment describes one example where the user enters the symptoms to the interview screen indicated by the food proposing system. However, the food proposing system may receive immediately or afterward measurements from equipments such as a body composition meter, a blood-pressure meter, a pedometer, a sleep meter used daily by the user without imposing a burden on the user. Furthermore, if medical checkup result and interview result that have been acquired by face-to-face interview mainly with a doctor or the like are available, the food proposing system may receive the result data from the device that stores the result data.

The food means natural food or processed product thereof that are available in the market to the user. Generally, the user acquires multiple foods, and then cooks in his/her own way to ingest the cooked food as a menu. The food is in a pre-stage before cooking, and it is different from the menu that is in a post-stage after cooking. For example, carrots, peppers and onions are the food. The user can cook these foods to prepare menus such as fried rice, stir-fried vegetables and the like. Moreover, plain yogurt is produced by processing in a factory the milk that is the natural food. This processing is not performed by the user. Thus, both the milk and the plain yogurt can be categorized into the food.

The nutrient is chemical substance that can be ingested daily by the user and the effect degree thereof for improving the symptoms and the content thereof in the food can be measured. The nutrient may include not only proteins, potassium, calcium and the like that are nutrient composition for which the legal obligation to indicate is imposed but also lactic acid bacteria, digestive enzymes, citric acid and the like.

### [Food Proposing System]

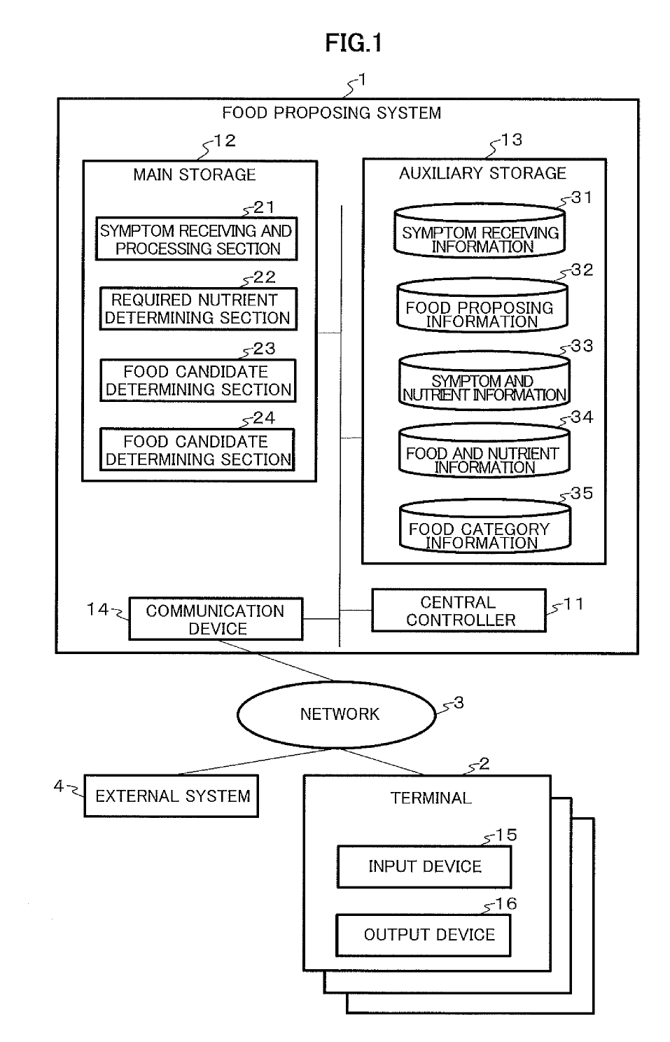

With reference to FIG. 1, a configuration of a food proposing system 1 will be described. The food proposing system 1 is a common computer. The food proposing system 1 includes a central controller 11, main storage 12, an Auxiliary storage 13, and a communication device 14. The food proposing system 1 may also include an input device and an output device (not illustrated). These are connected to each other via a bus. The Auxiliary storage 13 stores symptom reception information 31, food proposing information 32, symptom and nutrient information 33, food and nutrient information 34, and food group information 35 (the details of which will be described below). A symptom receiving and processing section 21, required nutrient determining section 22, food candidate determining section 23, and food candidate indicating and processing section 24 that are in the main storage 12 are programs. Hereinafter, when a subject is described as “◯◯ section is/does”, the central controller 11 reads the ◯◯section from the Auxiliary storage 13 to load the program of the Auxiliary storage into the main storage 13, achieving the function of the ◯◯ section (the details of which will be described below).

A terminal 2 can communicate with the food proposing system 1 via a network 3. The terminal 2 is a common portable computer. The terminal 2 includes an input device 15 and an output device 16. The input device 15 and the output device 16 may be an integral input and output device such as a touch screen. The terminal 2 can also communicate with an external system 4 via the network 3. The external system 4 is, for example, the system (operated by a food supplier or the like) that outputs an example of the menu cooked using the received food.

### [Interview Screen]

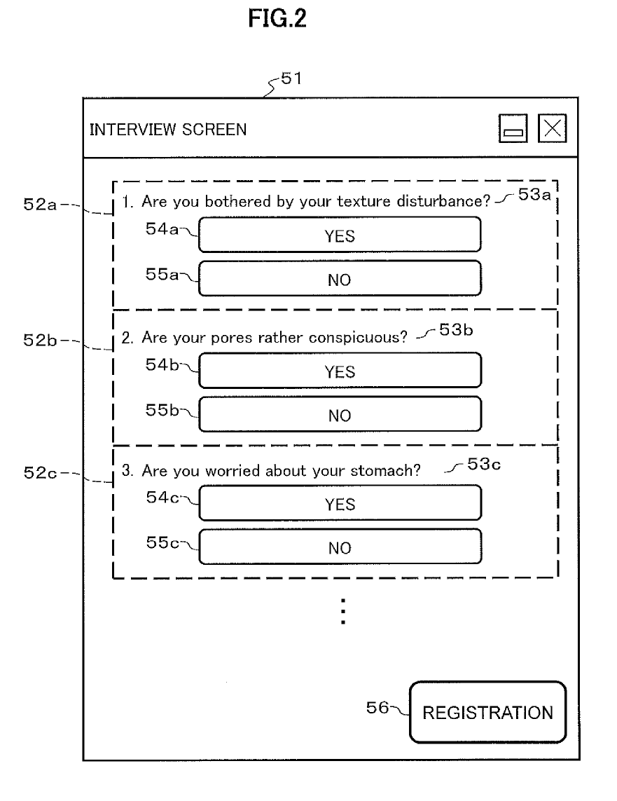

With reference to FIG. 2, an interview screen 51 will be described. The symptom receiving and processing section 21 of the food proposing system 1 displays the interview screen 51 to the output device 16 of the terminal 2. The interview screen 51 includes interview fields 52a, 52 b, 52c . . . . The Interview field 52a describes the question sentence to ask the user either presence or absence of the symptom “Are you bothered by your texture disturbance?” 53a, a “yes” button 54, and a “no” button 55a. The interview field 52b has the question sentence “Are your pores rather conspicuous?”, the “yes” button 54a and the “no” button 55a. The interview field 52c has the question sentence “Do you have an upset stomach?” 53c, the “yes” button 54c and the “no” button 55c.

The symptom receiving and processing section 21 receives either of the “yes” button or the “no” button pressed by the user with respect to each of the question sentences. When the user presses a “registration” button 56, the symptom receiving and processing section 21 prepares a new record of the symptom reception information 31 (the details of which will be described below).

### [Food Proposing Screen]

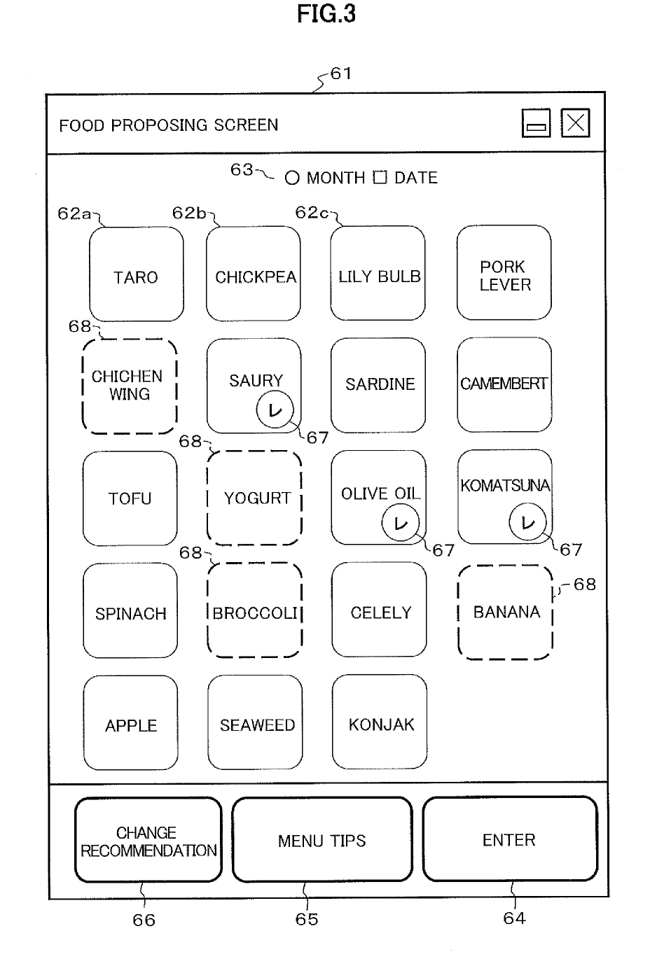

With reference to FIG. 3, a food proposing screen 61 will be described. The food candidate processing part 24 of the food proposing system 1 displays the food proposing screen 61 to the output device 16 of the terminal 2. The food proposing screen 61 has food icons 62a, 62b, 62c, . . . , a reference time 63, an “Enter” button 64, a “menu tips” button 65 and a “Change recommendation menu” button 66. The food icon 62a and the like indicate the food that may improve the symptom(s) described in the question sentence(s) corresponding to “yes” that has (have) been input in the interview screen 51 (FIG. 2).

The food candidate indicating and processing section 24 receives arbitrary one or more food icons pressed (temporarily selected) by the user. Next, the food candidate indicating and processing section 24 puts a temporary selection mark 67 to the food icon. When the food candidate indicating and processing section 24 receives the food icon to which the temporary selection mark 67 is put, pressed (temporarily selected) by the user, the food candidate indicating and processing section 24 deselects the food icon to remove the temporary selection mark 67.

Subsequently, the food candidate indicating and processing section 24 receives the “Entry” button 64 pressed (the selection is confirmed) by the user with at least one of the foods being temporarily selected. Then, the food candidate indicating and processing section 24 makes the food icon to which the temporary selection mark 67 is put a confirmation state (for example, highlighted as indicated by a sign 68). Next, the food candidate indicating and processing section 24 stores all of the indicated foods and the foods that have been selected and confirmed in food proposing information 32 (the details of which will be described below). After that, the user can temporarily select the food other than the foods that have been already selected to be confirmed, next can also confirm the temporarily selected food additionally.

The food candidate indicating and processing section 24 receives the “menu tips” button 65 pressed by the user with at least one of the food being selected and confirmed (or temporarily selected). Then, the food candidate indicating and processing section 24 gets access to the external system 4 via the network 3. Next, the food candidate indicating and processing section 24 retrieves the example of the menus using the foods that have been already selected and confirmed (or temporarily selected) to display the retrieved menu to an arbitrary field on the food proposing screen 61. The reference time 63 may be either a present date or a freshness date by which the user should finish ingesting the proposed food (e.g., 3 days after the present date). The “Change recommendation menu” button 66 will be described in details below.

### [Symptom Reception Information]

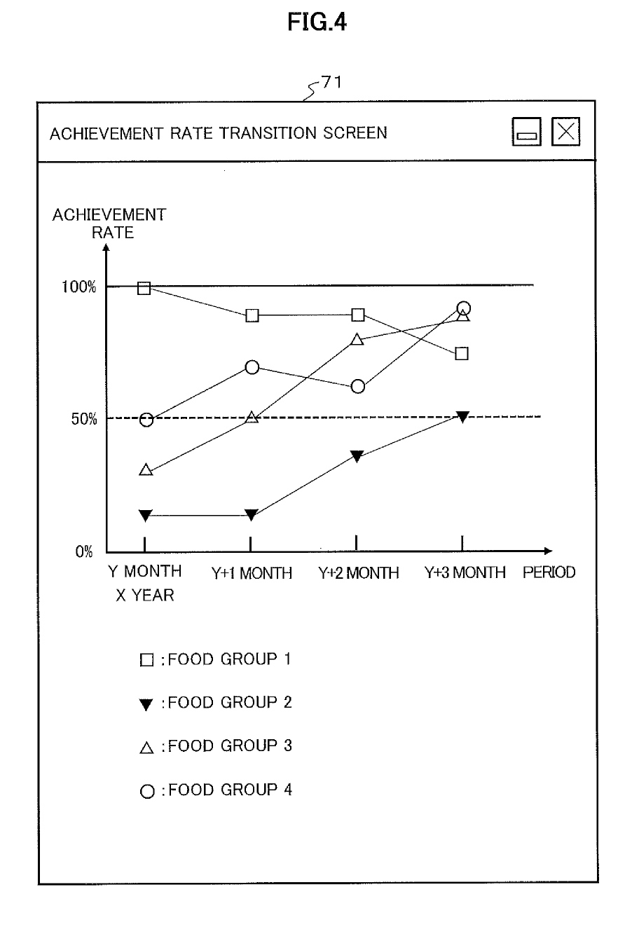

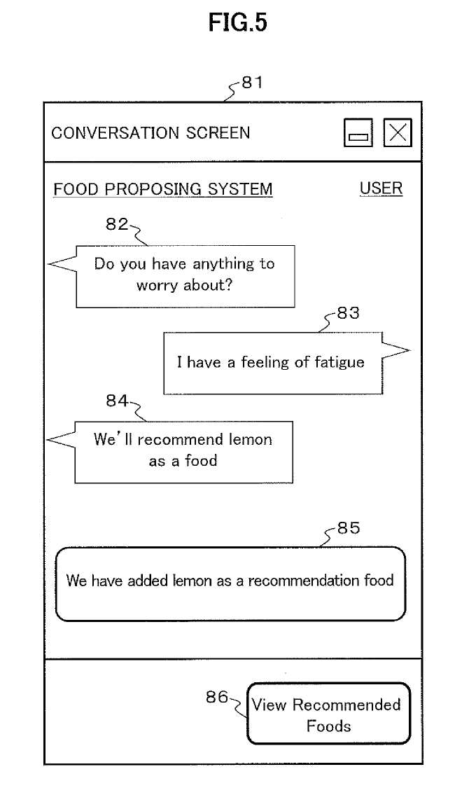

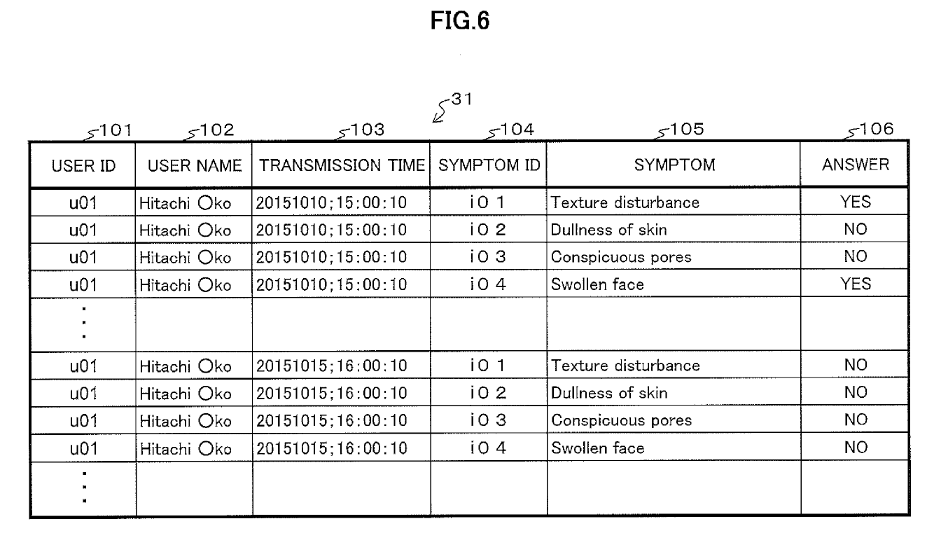

For convenience of explanation, while postponing the description of FIGS. 4 and 5, with reference to FIG. 6 above, the symptom reception information 31 will be described earlier. The symptom reception information 31 includes an ID name, a transmission time, a symptom ID, symptom, and an answer stored, in association with an user ID, in an user ID field 101, in a user name field 102, a transmission time field 103, a symptom ID field 104, a symptom field 105, and an answer field 106 respectively.

The user ID in the user ID field 101 is the identifier that may uniquely specify a user. The user may be a food proposing system 1 user and also be a terminal 2 user.

The user name in the user name field 102 is a full name of the user.

The transmission time in the transmission time field 103 is year, month, day, hour, minute and second of the time at which the user presses the “registration” button 56 (FIG. 2).

The symptom ID in the symptom ID field 104 is the identifier that uniquely specifies a symptom.

The symptom in the symptom field 105 is the above-described symptom. It should be noted that the question sentence is originally an question sentence, for example, “Are you bothered by your texture disturbance?”. Here, for simplicity and understanding, an affirmative sentence “My texture of skin is disturbed” is described as the symptom.

The answer in the answer field 106 is either “yes” or “no”.

### [Food Proposing Information]

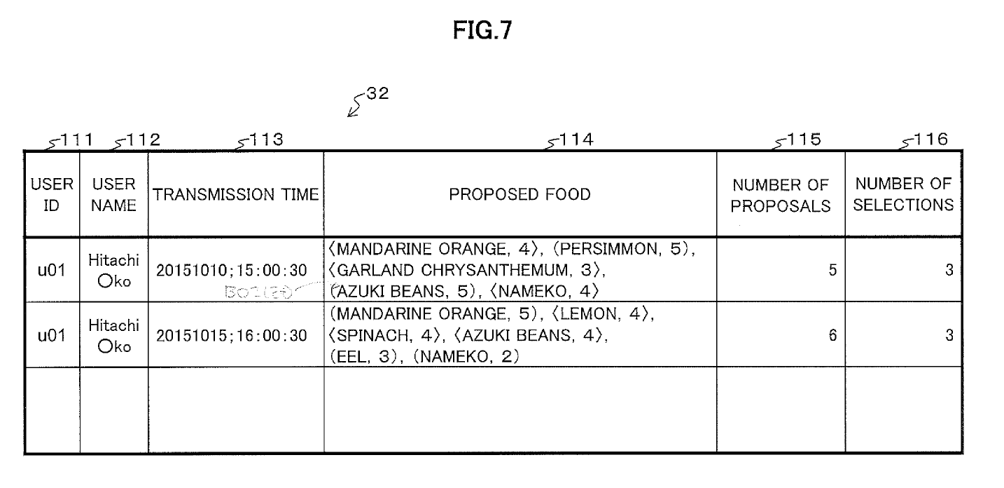

With reference to FIG. 7, the food proposing information 32 will be described. The food proposing information 32 includes a user name, a transmission time, proposed food, the number of proposals, and the number of times of selection, in association with a user ID stored in the user ID field 111, in a user name field 112, a transmission time field 113, a proposed food field 114, a number of proposals field 115, and a number of selections field 116 respectively.

The user ID in the user ID field 111 is the same as the user ID in FIG. 6.

The username in the user name field 112 is the same as the user name in FIG. 6.

The transmission time in the transmission time field 113 is year, month, day, hour, minute and second of the time at which the user presses the “Entry” button 64 (FIG. 3).

The Proposed food in the proposed food field 114 is an combination of a food and a score thereof (detailed below), the food being indicated by the food candidate indicating and processing section 24, as a food icon 62a or the like in the food proposing screen 61 (FIG. 3). The combination of the food and the score is enclosed in “( )” or “< >”. “( )” indicates that the food was indicated not to have been selected and confirmed by the user. “< >” indicates that the food was indicated to be selected and confirmed by the user.

The number of proposals described in a number of proposal field 115 is the number of foods proposed (indicated), that is, the number of “( )” and “< >”.

The number of times of selection described in a number of selection field 115 is the number of foods selected and confirmed by the user, that is, the number of “< >”.

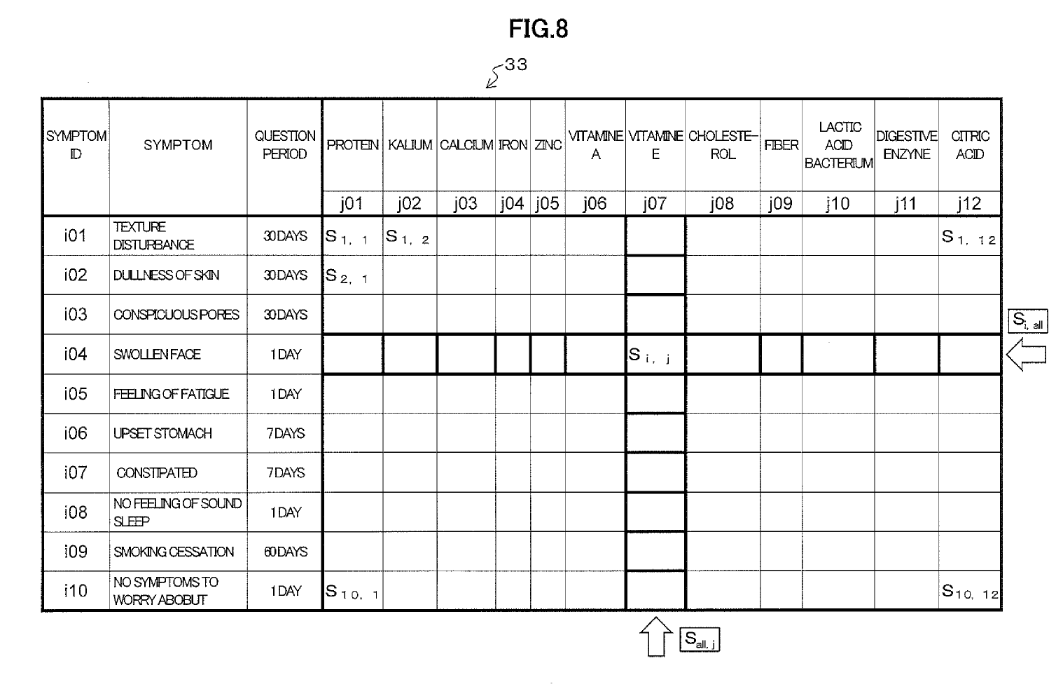

With reference to FIG. 8, the symptom and nutrient information 33 will be described. The symptom and nutrient information 33 includes a matrix in which symptoms are indicated on a vertical axis, and nutrients are indicated on a horizontal axis. Further, a cell arranged at the intersection between the vertical axis and the horizontal axis has a value to indicate the effect degree of the nutrient for improving of the symptom stored. More specifically, the symptom ID is indicated on the vertical axis along with the symptom. The symptom ID and symptom here is the same as the symptom ID and symptom of FIG. 6. Though there are 10 kinds of symptoms in total in FIG. 8, the user can set the number arbitrarily. Additionally, “question period” will be described below. A nutrient ID (j01, j02, . . . ) is arranged along with the nutrient (protein, potassium . . . ) on the horizontal axis. The nutrient ID is the identifier that uniquely specifies the above-mentioned nutrient. Though there are 12 kinds of nutrients in total in FIG. 8, the user can set the number arbitrarily. Needless to say, there actually exists other nutrients that are effective for improving respective symptoms besides the 12 kinds of nutrients as illustrated in FIG. 8 (FIG. 8 is a simplified table for illustration).

A matrix component of 10 rows and 12 columns can be represented as “S_{i, j}” (i=1, 2, . . . , 10, j=1, 2, . . . , 12). The vector consisting of all components (effect degree values) of any of the rows in the symptom and nutrient information 33 is represented as “S_{i, all}”. Similarly, a vector consisting of all components of any of the columns in the symptom and nutrient information 33 can be represented as “S_{all, j}”. The details of which will be given later.

### (Question Period)

Time scale by which the symptom changes is different depending on the symptom. For example, physical disorder (hangover, etc.) due to drinking is improved at least next day or two days later. Thus, it is meaningful that the question “Do you still have physical disorder due to drinking?” is indicated by the symptom receiving and processing section 21 every day. On the other hand, it takes about one week to improve constipation, about one month to improve skin symptoms, and a few months to improve high blood pressure.

Therefore, the symptom and nutrient information 33 includes the question period in association with the symptom. The symptom receiving and processing section 21 is not intended to display the present question after indicating last time the question asking either presence or absence of the symptom and until the number of days of the question period has elapses. Alternatively, the symptom receiving and processing section 21 highlights (displays by default) the button of last answer out of “yes” button and “no” button until the number of days of the question period elapses. Further, only when the symptom changes, the symptom receiving and processing section 21 may receive the other button pressed by the user.

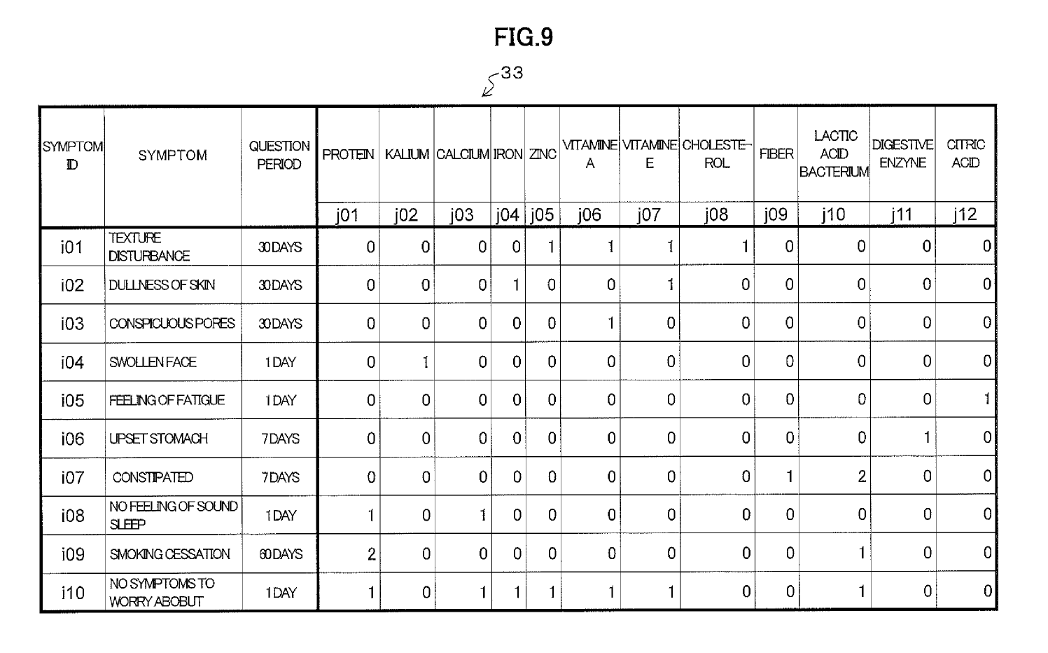

FIG. 9 is also the symptom and nutrient information 33. However, FIG. 9 has a specific value of the “S_{i, j}” stored in the cell arranged at each of the respective intersections. This value is the effect degree. The effect degree may take positive and negative values and “0”. As the simplified example, the effect degree can be either one of the following four values. FIG. 9 also follows this example, unintentionally having no description of “2” indicates the nutrient is significantly effective for improving the symptom.

“1” indicates the nutrient is effective for improving the symptom.

“0” indicates the nutrient is ineffective for improving the symptom.

“−1” indicates the nutrient has a harmful for improving the symptom.

### [Food and Nutrient Information]

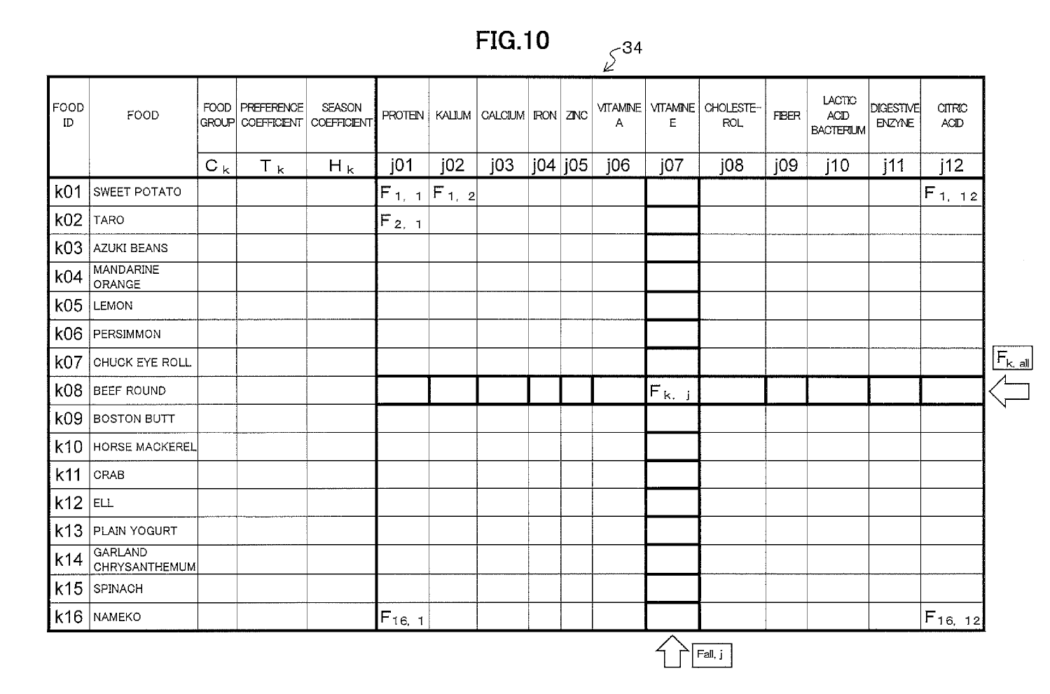

With reference to FIG. 10, the food and nutrient information 34 will be described. The food and nutrient information 34a includes the matrix in which foods are indicated on a vertical axis, nutrients are indicated on a horizontal axis. Further, the cell arranged at the intersection between the vertical axis and the horizontal axis has the value to represent the content of the nutrient in the food stored therein. More specifically, the food ID is aligned on the vertical axis with the food. The food ID is the identifier that uniquely identifies a food. Though there are sixteen foods in FIG. 10, the user can set the number arbitrarily. Additionally, “food group”, “preference coefficient” and “season coefficient” will be described later. The nutrient ID is aligned on the horizontal axis in FIG. 10 with the nutrient as in FIG. 8 and FIG. 9. Though FIG. 10 has twelve nutrients in total, the user can set the number arbitrarily. Needless to say, the respective foods may actually include other nutrients other than the twelve foods illustrated in FIG. 10 (FIG. 10 is a simplified table for illustration). In the present embodiment, it is assumed that the vertical axis on which the symptom and nutrient information 33 (FIG. 8, FIG. 9) is indicated and the horizontal axis on which the food and nutrient information 34 (FIG. 10, FIG. 1) is indicated completely corresponds to each other in a number, a kind and a sequence of the nutrients.

A matrix component of 10 rows and 12 columns of FIG. 10 can be represented as “F_{k, j}” (k=1, 2, . . . 10, j=1, 2, . . . 12). A vector consisting of all components (content value) of any of the rows in the food and nutrient information 34 is represented as “S_{k, all}”. The details of which will be given later. Similarly, a vector consisting of all components of any of the columns in the symptom and nutrient information 34 can be represented as “S_{all, j}”. Additionally, though “S_{all, j}” has been mentioned here to explain the structure of the matrix, “S_{all, j}” will not be used hereinafter in the present embodiment.

With attention being paid again to FIG. 10, the food and nutrient information 34 includes a food group Cₖ, a preference coefficient Tₖ and a season coefficient Hₖ in association with the food.

### [Season Coefficient]

With respect to the food as a natural product, it is possible to define the season (period) when the food is readily available and the season when the food is not readily available, from the viewpoint of natural condition. The season when the food is readily available from the viewpoint of the natural condition is called a season for the food. The season is different depending on the food. Even in the case of the same food, the season is different depending on the region. Today, most of foods are available regardless of the season by progress of cultivation and aquaculture technologies. However, even in the case of the same food, difference is caused in quality spontaneously between the case where the food was grown naturally and the case where the food was artificially grown. The season coefficient Hₖ is the value having a range of, for example, “0≤Hₖ≤2”.

The Auxiliary storage 13 is assumed to store the season table (not illustrated) having the best season (e.g. a month) for the respective foods stored. Further, the food candidate determining section 23, if the present time is in the season, brings the season coefficient close to “2”. The food candidate determining section 23, if the present time is not in the season, brings the season coefficient close to “0”. In this way, the season coefficient is changed constantly with “1” as a reference. However, the processed food that is not influenced by season has the season coefficient fixed near “1” to be hardly changed.

### [Preference Coefficient]

The value obtained by dividing the number of times a particular user confirmed the selection of a particular food during a certain period of time by the number of times that the food has been proposed to the user (indicated to the food proposing screen 61) during the period is defined as “selection rate”. The preference coefficient Tₖ has a range of, for example, 0≤Tₖ≤2 depending on the selection rate. Specifically, the initial value of the preference coefficient is “1”. The food candidate determining section 23 constantly monitors the selection rate. If the selection rate exceeds a predetermined threshold value, the food candidate determining section 23 multiplies the preference coefficient by, for example, “1.2” (the upper limit value is “2”). Similarly, if the selected rate is less than a predetermined (different) threshold value, the food candidate determining section 23 multiplies the preference coefficient by, for example, “0.8”. As a special case, if the user is prohibited or restricted from ingesting the food by the judgment of a doctor and the like (because the food is allergic for the user), the preference coefficient is fixedly set to be “−1”. Though FIG. 10 directly shows that, it should be noted that the preference coefficient is defined for the combination of the food, the user and the period in this way.

### [Food Group]

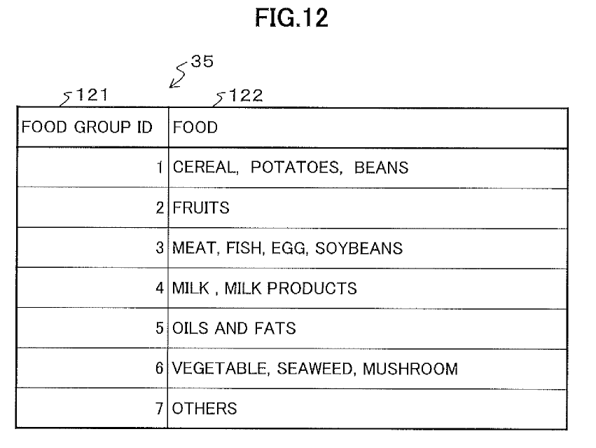

The food candidate determining section 23 proposes the user the food containing the nutrient that is effective for improving the symptom. However, if the user ingests only the same food, he/she loses nutritional balance of the foods, so that the risk of ruining his/her health is increased. To prevent this unbalance, the foods having a common nutrient may be classified into several food groups, so that at least one of the foods can be proposed from each of the food groups. The food groups will be described later as explanation for FIG. 12.

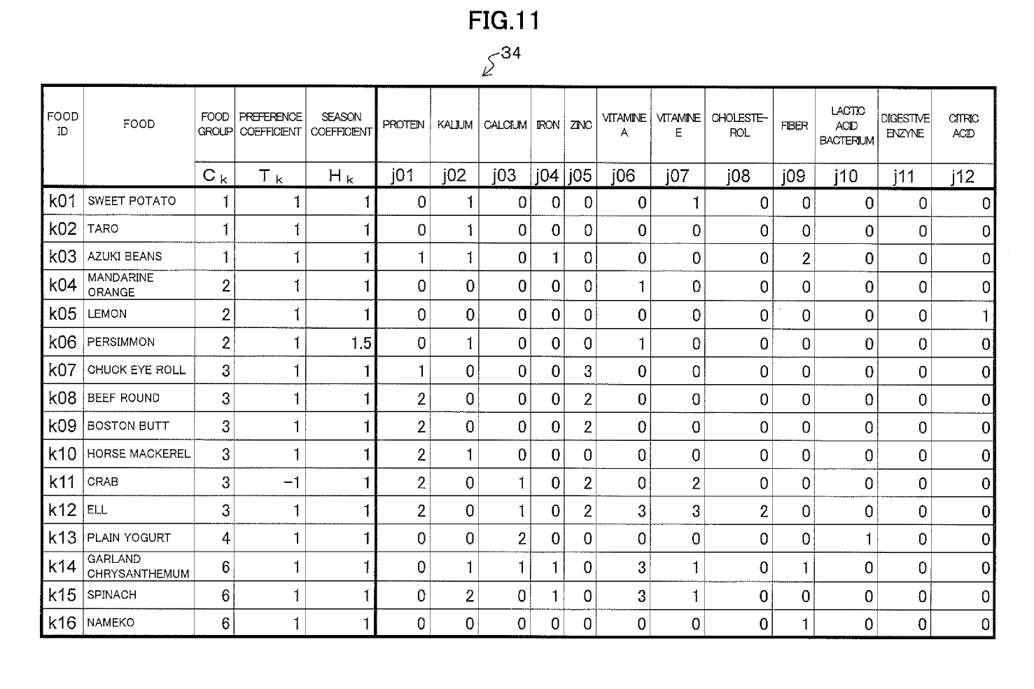

Similarly, FIG. 11 is the food and nutrient information 34. However, in FIG. 11, the cell at each intersection has a specific value for “F_{k, j}” stored. This value indicates the content. The content may take a positive value and “0”. As a simplified example, the content is either one of the following four values. FIG. 11 also follows this example.

“3” indicates that the food contains especially a large amount of the nutrient.

“2” indicates that the food contains a large amount of the nutrient.

“1” indicates that the food contains the nutrient.

“0” indicates that the food does not contain the nutrient.

### [Effect Degree and Content]

The content of the nutrient is uniquely defined for a certain food. For example, “sweet potato” has an approximately specific chemical composition (though being subtly depending on natural condition of the year when the food was gathered), and the contents of the respective nutrients may be considered to be approximately specific. On the other hand, the effect degree for improving a certain symptom is not necessarily defined uniquely. The effect degree may vary depending on the physical constitution of each of the users, and the physical constitution of the human population under a certain geographic condition.

For example, if a user “u01” has the symptom “texture is disturbed”, “vitamin A” is effective while “vitamin E” is not effective. However, for example, if a user “u02” has the same symptom “texture is disturbed”, “vitamin E” may be effective while “vitamin A” may not be effective (this example is just for explanation, thus physiologically verification has not been performed). In other words, the each of the effect degrees (S) of the symptom and nutrient information 33 depends on the physical constitution of the individual or the human population, thus it is important for the provision of appropriate foods and updates the respective effect degrees to their optimal values (Food group information).

With reference to FIG. 12, the food group information 35 will be described. In the food group information 35, the food group is stored in a food group field 122 in association with a food group ID stored in a food group ID field 121.

The food group ID in the food group ID field 121 is the identifier that specifies uniquely the food group. Here, numbers from 1 to 7 are assigned to the respective food groups.

The names of the food groups are described in the food group field 122. These names make it possible to know what kinds of the foods are included in the respective food groups.

The food belongs to either one of the food groups. It is preferable that the user ingests at least one food from each of the food groups from “1” to “7”. Further, the degree (size) of subdivision of the food groups depends on the setting by the user. For example, “fruit” may be further classified by producing districts, and “oils and fats” may be further classified by animal and vegetable.

### [Processing Procedure]

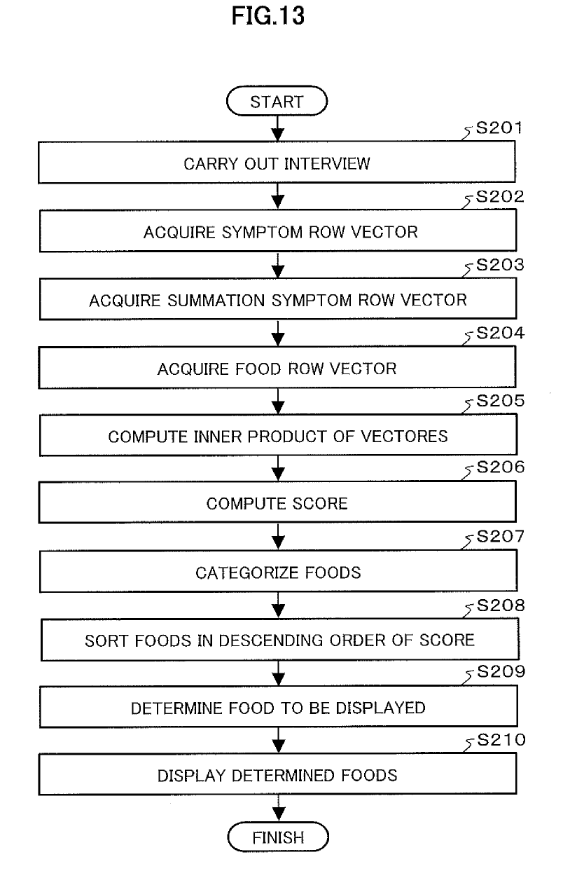

With reference to FIG. 13, a processing procedure will be described.

In S201, a symptom receiving and processing section 21 of the food proposing system 1 executes an interview. Specifically, first, the symptom receiving and processing section 21 displays an interview screen 51 (FIG. 2) to the output device 16 of the terminal 2. At this time, the symptom receiving and processing section 21 refers to a question period field of the symptom and nutrient information 33 so as not to display the question the days of the question period of which doesn't pass after the question asking either presence or absence of a symptom was displayed last time up to the present time. Alternatively, the symptom receiving and processing section 21 displays by default either of the “yes” button or the “no” button that is a previous answer.

Second, the symptom receiving and processing section 21 receives either of the “yes” button or “no” button pressed by the user for each of the interview fields 52a or the like and subsequently pressing of the “register” button 56. The symptoms reception processing part 21 receives at this time from the terminal 2 the user name, the user ID and the time at which the “registration button” 56 has been pressed.

Third, the symptom receiving and processing section 21 prepares a new record of the symptom reception information 31 (FIG. 6) on the basis of the data received in the “second” process in S201 to store the prepared record therein. The number of records stored at this time is equal to the number of the interview fields (the number of questions).

In S202, the required nutrient determining section 22 of the food proposing system 1 retrieves a symptom row vector. Specifically, first, the required nutrient determining section 22 retrieves, out of the records of the symptom reception information 31 stored in the “third” process of step S201, the symptom ID of the record the answer field 106 of which describes “yes”.

Second, the required nutrient determining section 22 searches the symptom and nutrient information 33 (FIG. 9) for the corresponding vector “S_{i, all}” using the symptom ID retrieved in the “first” process In S20 as a search key. The vector retrieved at this time is referred to as “symptom row vector”. The number of the symptom row vectors is equal to the number of “yes” buttons pressed by the user.

In S203, the required nutrient determining section 22 retrieves a summation symptom row vector. Specifically, the required nutrient determining section 22 sums up respective components of the symptom row vectors retrieved in “the second” process in S202 to define the sum obtained as the result as the summation symptom row vector.

Now, when the summation symptom row vector is represented as “ΣΣS_{i,all}” an equation 1 in the following is satisfied.

ΣΣS_{i,all}=αS_{i,all}+βS_{i,all}+γS_{i,all}+ . . . (Equation 1)

Here, a subscript “i” of each item in the right side of the equation 1 corresponds to the symptom to which the “yes” button has been pressed. For example, if there are 3 symptoms to which the “yes” button has been pressed as follows: “texture is disturbed” (symptom ID is i01), “pores are rather conspicuous” (symptom ID is i03) and “is constipated” (symptom ID is i07), the right side of the equation 1 is “αS_{1,all}+βS_{3,all}+γS_{7,all}”. α, β, γ, . . . respectively is the weight which is set in advance by the user for each of the symptoms, and may take a positive value. The description will be continued by assuming that α=β=γ= . . . =1 in the following description for simplicity. However, the user who wants to place importance on, for example, “texture is disturbed” can also set the weight that is to be multiplied to “S_{1,all}” to be “2”, the other weights to be “1”.

In S204, the food candidate determining section 23 of the food proposing system 1 retrieves the food row vector. Specifically, the food candidate determining section 23 retrieves the food group preference coefficient, the season coefficient and the vector “F_{k, all}” of any one of the unprocessed records in the food and nutrient information 34 (FIG. 11). The vector retrieved at this time is referred to as a “food row vector”.

In S205, the food candidate determining section 23 computes an inner product of the vectors. Specifically, the food candidate determining section 23 computes the inner product of the summation symptom row vector retrieved in S203 and the food row vector retrieved in S204. The inner product is the scalar value obtained by the totalizing the products of each component of two vectors.

In S206, the food candidate determining section 23 computes the score. Specifically, the food candidate determining section 23 multiplies the product computed in S205 by the preference coefficient retrieved In S204. The food candidate determining section 23 further multiplies the multiplication result by the season coefficient retrieved in S204 to obtain the score.

The food candidate determining section 23 repeats the processing from S204 to S206 for each of the records in the food and nutrient information 34 (FIG. 11). In the repetition processing, the food candidate determining section 23 computes the score for the respective foods. The examples of data stored in the food candidate determining section 23 when the repetition processing is finished are as follows.

(Food, score)=(sweet potato, 5), (taro, 4), (boiled red bean, 6), (mandarin orange, 2), (lemon, 1), (persimmon, 3), (Chuck eye roll, 3), (beef round, 2), (Boston butt, 4)

The data is referred to as “food score data”.

In S207, the food candidate determining section 23 classifies the foods. Specifically, the food candidate determining section 23 classifies, on the basis of the food groups retrieved In S204, the food score data according to the respective food groups. At the stage when the classification is finished, the food score data is as follows.

(Food, score)₁=(sweet potato, 5), (taro, 4), (boiled red beans, 6)

(food, Score)₂=(mandarin orange, 2), (lemon, 1), (persimmon, 3)

(food, scores)₃=(Chuck eye roll, 3), (beef round, 2), (Boston butt, 4)

Here, the subscripts “1”, “2” and “3” are the food group IDs. There are 7 food group IDs in FIG. 12. However, for simplification, it is assumed in the following there are 3 food group IDs.

In S208, the food candidate determining section 23 sorts the foods in the descending order of the scores. At the stage when this sort is finished, the food score data is as follows.

(Food, score)₁=(boiled red beans, 6), (sweet potato, 5), (taro, 4)

(food, Score)₂=(persimmon, 3) (mandarin orange, 2), (lemon, 1) (food, scores)₃=(Boston butt, 4), (Chuck eye roll, 3), (beef round, 2)

In S209, the food candidate determining section 23 determines the food to be indicated. Specifically, first, the food candidate determining section 23 retrieves a number of candidates by food group number of candidates by food group is data such as (food group ID, the number of candidates)=(1,2), (2,2), (3,2). The food candidate determining section 23 receivers the number of food candidates proposed to the user himself/herself preset by the user to store the data in the Auxiliary storage 13 as the number of candidates by food group. In the above example for the number of food candidates, it is shown that the user hopes that 2 foods will be proposed respectively from each of the food group “1”, the food group “2” and the food group “3”.

Second, the food candidate determining section 23, on the basis of the number of candidates by food group retrieved in the “first” in S209, specifies the food candidates as many as the number of which is equal to the number of candidates, in the descending order of the score. At the stage when this specification is finished, the food score data is as follows.

(Food, score)₁=(boiled red beans, 6), (sweet potato, 5)

(Food, Score)₂=(persimmon, 3) (mandarin orange, 2)

(Food, score)₃=(Boston butt, 4), (Chuck eye roll, 3)

In S210, the food candidate indicating and processing section 24 of the food proposing system 1 displays the food. Specifically, first, the food candidate indicating and processing section 24 displays the food proposing screen 61 (FIG. 3) to the output device 16 of the terminal 2. More specifically, boiled red beans, sweet potato, persimmon, mandarin orange, Boston butt, Chuck eye roll are indicated as the food icons. The food candidate indicating and processing section 24 may arrange the food icons by the food group and in the descending order of the score.

Second, the food candidate indicating and processing section 24 receives the selection of the food confirmed by the user. Now, it is assumed that the user confirms the selection of “sweet potato”, “mandarin orange” and “Chuck eye roll” out of the six food icons. In this case, the food candidate indicating and processing section 24 receives, from the terminal 2, the user name, the user ID and the time at which the user ID and “Entry” button 64 was pressed.

Third, the food candidate indicating and processing section 24 prepares anew record of the food proposing information 32 (FIG. 7) on the basis of the data received in the “second” in S210 to store the new record therein. In the above example, the food candidate indicating and processing section 24 stores in the new record “(boiled red beans, 6), (sweet potato, 5), (persimmon, 3), (mandarin orange, 2), (Boston butt, 4), (Chuck eye roll, 3)” as the proposed foods.

Afterwards, the processing procedure is finished.

### [Modification 1: Random Candidate Determination]

In the above description, the food candidate determining section 23 determines the food candidate to be proposed to the user in the descending order of the score. However, when such a strict rule is applied, the same combination of the foods is apt to be proposed for the input symptom every time. Therefore, the food candidate determining section 23 may execute the processing of first pattern or second pattern in the following.

### [First Pattern]

Retrieving number p of foods having the score equal to or greater than a predetermined threshold, from each of the food groups.
Retrieving at random, from among the number p of foods, the foods as many as the number of which is equal to the number q of candidates by food group (q<p).

According to the first patter, it is possible to prevent only one kind of the foods to be retrieved as a candidate from among the foods with similar scores every time.

### [Second Pattern]

Extracting at random the foods as many as the number of which is equal to the number of candidates by food group from among the each of the food groups to prepare a set of the food candidates including the candidates belonging to different kinds of the food groups. A plurality of the sets will be retrieved
Totalizing the scores of the foods included in each of the sets to determine the set having the largest total score as the food candidate to be proposed to the user.

According to the second pattern, even the food having a low score can be a candidate with the help other foods having larger scores.

If the user views the food proposing screen 61 (FIG. 3) to have impressions such as “there are few favorite foods” and “similar foods are often indicated”, the food candidate determining section 23 receives the “Change recommendation” button 66 pressed by the user to execute the first pattern or the second pattern. Then, the food candidate indicating and processing section 24 displays the food icon for the food determined as the result. Accordingly, some of the food icons are replaced by other food icons to prevent the user from getting bored.

### [Second Modification: Candidate Determination on the Basis of Probability]

Even if the food has a small score, as the measure for increasing the probability that the food will be selected as the candidate to be indicated, the food candidate determining section 23 may execute the processing of third pattern in the following.

### [Third Pattern]

Adding to the score a predetermined positive value significantly greater than the value of the score to obtain a corrected score

For example, if the score of sweet potato is “2”, “100” is added to the “2”, “102” is defined as the corrected score.
Determining the food candidate to be indicated on the basis of the probability proportional to the ratio of the corrected score of the food to the total of the corrected scores of all of the foods

If the score of the taro is “6” and the score of the sweet potato is “2”, the sweet potato is less likely to be selected as the food candidate than the taro (the difference in the score is 6−4=2). According to the third pattern, the corrected score “106” of the taro, while the corrected score of the sweet potato is “102”. In addition, if the total of the corrected scores of all of the foods is “620”, the taro and sweet potato can be selected as the food candidate with probabilities of “0.171=106/620” and “0.165=102/620” respectively. Since the difference in the probability of the selection is 0.171−0.165=0.006, the sweet potato is more likely to be selected as the candidate.

### [Third Modification: Having No Symptoms to Worry About]

Even if symptom to be improved exists objectively, the user is often unaware of the symptom. In this case, the user may answer “yes” to the question “Do you mean that you have no symptoms to worry about?”. However, it is necessary for improving the hidden symptom to propose to such a user some food candidates on the basis of some grounds. Therefore, how to determine respective components of a row vector “S_{10, all}” for “i10” in FIG. 9 becomes an issue. Therefore, the required nutrient determining section 22 may execute the processing of a fourth pattern in the following.

### [Fourth Pattern]

Summing up all components of a column vector “S_{all, k}” for each of the nutrients (where, “all” doesn't include “10”) in FIG. 9
Specifying a predetermined (e.g., “5”) number of the nutrients in the descending order of the total value
Generating a row vector “S_{10, all}”₁ in which the effect degree of the specified nutrients is defined as “1”. “1” is assigned to 5 out of 12 components of the vector “S_{10, all}”

### [Fourth Modification: “I am not Sure”]

The user cannot always judge easily either of “yes” or “no” to the question. Accordingly, an “I am not sure” button may be indicated additionally to each of the interview fields 52a and the like in the interview screen 51 by the symptom receiving and processing section 21. Then, required nutrient determining section 22 considers “I am not sure” to be the same as “yes” and then retrieves the symptom row vector. However, if the answer is “I am not sure”, the required nutrient determining section 22 multiplies each component of the symptom row vector by t (0<t<1). If the user believes that he or she is more cautious than others, t is set to be a larger value. Otherwise, t is set to be smaller value.

[Fifth Modification: Point Provision after “Finish”, Etc.]

Selection and confirmation of all of the foods that have been proposed as the candidates by the user is referred to as “complete”. When the user completes, the ratio (achievement ratio) of the number of times of selection divided by the number of proposals that are included by the food proposing information 32 (FIG. 7) becomes 100%. The food candidate indicating and processing section 24 continuously monitors the achievement ratio, and when the food candidate indicating and processing section 24 detects that the user completes, the food candidate indicating and processing section 24 may execute either of the following processing.
Providing points to the user. The points as used herein may be, for example, the electronic money issued by the food producer (seller) that produces (sells) the foods that have been selected and confirmed
Adding next time and thereafter the foods that are included in the specific food group to the food candidates to be indicated. This food group, for example, may exclusively include “reward” foods such as sweets that may not especially affect the ingestion of other foods

### [Sixth Modification: Achievement Rate Transition Screen]

The fact that the achievement ratio V is gradually approaching 100% is visualized, even though the achievement ratio may not reach 100%, so that the user can easily maintain his/her motivation to complete. Accordingly, the food candidate indicating and processing section 24 may execute the following processing at any timing.
Computing the user achievement ratio, in time series and for each of the food groups, on the basis of the transmission time, the proposed food, the number of the proposals and the number of times of the selection included by the food proposing information 32 (FIG. 7), and the food group included by the food and nutrient information 34 (FIG. 11)
Indicating the transition of the computed achievement ratio to the output device 16 of the terminal 2 as an achievement ratio transition screen 71 (FIG. 4)

### [Seventh Modification: Conversation Screen]

The user may often have no time to answer carefully each of the questions. In addition, the user often remembers additionally a symptom to be answered “yes” immediately after the user answers briefly a plurality of the questions. The screen that can be used to make up for or save labor of the main interview screen 51 (FIG. 2) would be useful. Thus, the symptom receiving and processing section 21 or the like may execute the following processing.
Receiving by the symptom receiving and processing section 21 receives a predetermined instruction input by the user at any timing (pressing of “Conversation” button (not illustrated) or the like) to the input device 15 of the terminal 2
Indicating by the symptom receiving and processing section 21 a conversation screen 81 (FIG. 5) to the output device 16 of the terminal 2. The conversation screen 81 has a balloon “Do you have anything to worry about?” 82 indicated thereon
Receiving by the symptom receiving and processing section 21 the symptoms input by the user in an arbitrary way. Here, the user may select one or more symptoms from among choices of the symptoms indicated by the symptom receiving and processing section 21, and may also input text or voice of the symptom. The symptom receiving and processing section 21 displays the input result, for example, as a balloon “I have a feeling of fatigue” 83.
Retrieving by the required nutrient determining section 22, in accordance with the above-described processing, the symptom row vector
Determining by the food candidate determining section 23, in accordance with the above-described processing, the food to be indicated. However, the user may often be in such a situation that he or she cannot view the screen carefully. Thus, the number of foods to be indicated should be as small as possible (the number may be one).
Indicating by the food candidate indicating and processing section 24 the food, for example, as a balloon “We'll recommend lemon as recommendation food” 84.
Indicating by food candidate indicating and processing section 24 an attention-drawing message “We have added Lemon to recommended food” 85, in a case where a predetermined period of time hasn't passed since the food candidate indicating and processing section 24 displayed the food candidate proposing screen 61 (FIG. 3). In addition, when the user presses “View recommended food” button 86, the food candidate indicating and processing section 24 transits the screen to the food proposing screen 61 (FIG. 3). The food proposing screen 61 at a transition destination has the food icon “lemon” indicated thereon along with the food icon that has just been indicated (not illustrated in FIG. 3).

### [Eights Modification: Learning of Effect Degree]

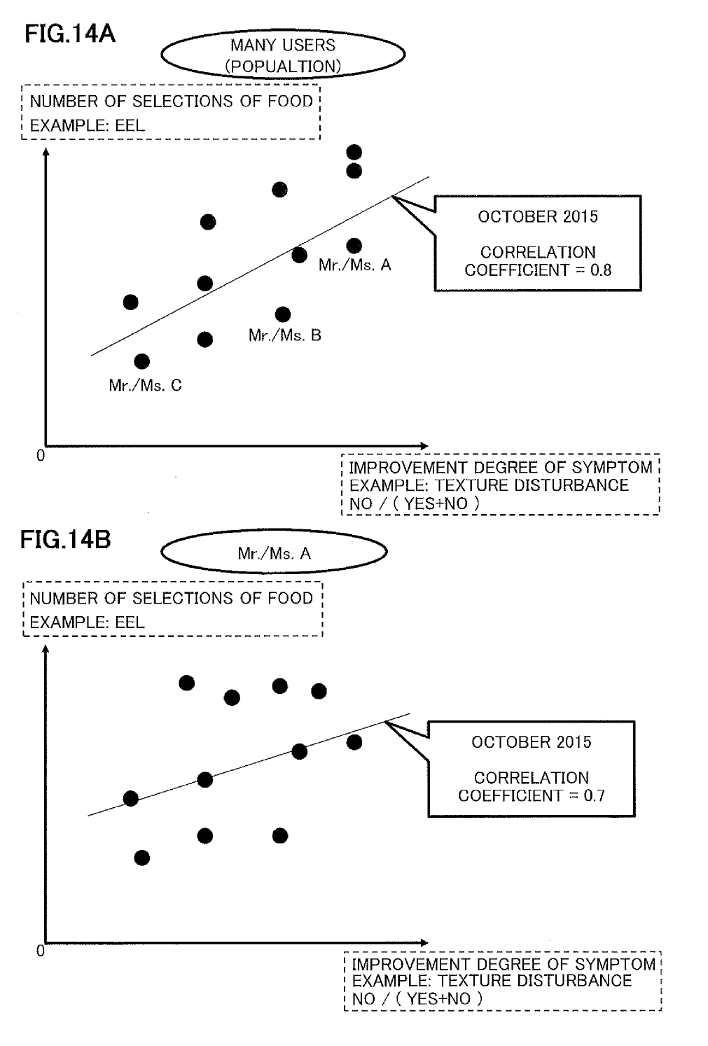

As described above, the symptom and nutrient information 33 (FIG. 9) represents physical constitution itself of the user (or user group). The effect degree (S_{i, j}) of the symptom and nutrient information 33 should be updated appropriately to ensure the provision of the appropriate foods. Accordingly, the required nutrient determining section 22 executes the following processing every a predetermined period (e.g., every month-end) executes the following processing through batch processing and the like.
Aggregating by the required nutrient determining section 22, from the symptom reception information 31 (FIG. 6) and the food proposing information 32 (FIG. 7), the latest data, for example, the data for the last one month on all of the users belonging to a certain population. The Auxiliary storage 13 has the user attribute information (not illustrated) having a user address, age, gender, name of workplace, type of business (sales, office work, etc.) or the like stored in association with the user ID. Accordingly, the required nutrient determining section 22 can extract, for example, the data on the female sales personnel in company A.
Computing by the required nutrient determining section 22 the improvement degree of a certain symptom and the number of selection of a certain food for each of the users belonging to the certain population. The improvement degree of the certain symptom is, for example, the value that is obtained by dividing the number of “no” answered to the symptom by the sum of the number of “yes” and the number of “no” answered to the symptom. The greater the value is, the better the physical condition is.
Plotting by the required nutrient determining section 22 the graphics indicating the user, on a coordinate plane (see FIG. 14A) with the improvement degree of in the symptom indicated on the horizontal axis and the number of times of selecting the food indicated on a longitudinal axis. The number of the plotted graphics corresponds to, for example, the number of the female sales personnel in the company A. Subsequently, a correlation coefficient r between the improvement degree of in the symptom and the number of times of selecting the food is computed. As is well known, the correlation coefficient r may take the value in a range of “1≤r≤1”.

Repeating, by the required nutrient determining section 22, such a processing for all the combinations of the symptoms and foods. As a result, the correlation coefficient is retrieved for each of the combinations.
Specifying the combination of the symptom and the food with the correlation coefficient r in a predetermined range (e.g., 0.8≤r). For example, it is assumed that the combination of “texture is disturbed” and “eel” is specified.
Retrieving by the required nutrient determining unit 22 a row vector “F_{12,j}” for “eel” included in the food and nutrient information 34 (FIG. 11). Additionally, the food ID for the eel is “k12”.
Specifying by the required nutrient determining section 22 the nutrient the content of which is not indicated as “0” by the vector “F_{12, j}”

For example, it is assumed the nutrients “zinc” and “Vitamin E” are specified here.
Updating by the required nutrient determining section 22 the values of the effect degrees “S_{1,5}” and “S_{1,7}” to larger values in the symptom and nutrient information 33 (FIG. 9). For example, the existing value “1” is multiplied by “1.5” to be updated. In addition, the symptom ID for the symptom “texture is disturbed” is the “i01”, the nutrient id for the nutrient “zinc” is “j05”, and the nutrient ID for the nutrient “vitamin E” is “j07”.

More generally, the first and second threshold values for the correlation coefficient r are represented as rₛ₁ and rₛ₂ (−1<rₛ₁<0<rₛ₂<1) respectively. It is assumed the effect degree S may take “0”, and positive and negative values. Under this assumption, the corresponding relationship between the value of the correlation coefficient r and the way in which the effect degree

S is updated is as follows.
If −1≤r<rₛ₁ is established, the effect degree S_{i, j} is made smaller.
If rₛ₁≤r<rₛ₂ is established, the effect degree of S_{i, j} is not updated.
If rₛ₂ r≤1 is established, the effect degree S_{i, j} is made larger.

The required nutrient determining section 22 may execute the similar processing for a specific user (see FIG. 14 B). As a result of executing simultaneously the processing of FIG. 14A and FIG. 14B, the difference between the correlation coefficient of the population Mr./Ms. A belongs to and the correlation coefficient of Mr./Ms. A as an individual may exceed a predetermined threshold value. In this case, the required nutrient determining section 22 may output a warning message. The warning message for the terminal 2 of Mr./Ms. A's is “Your are slightly different in physical constitution from other people around you. We'll recommend that you receive the advice from an expert”. The warning message for the communication device of a manager's in a workplace is “Mr./Ms. A is slightly different in physical constitution from other people around Mr./Ms. A. Please advise Mr./Ms. A especially on his or her daily health management”.

### [Advantageous Effects of Embodiments]

The present embodiment exhibits the following effects.

(1) The user who attempts to cook and ingest the food in his/her own way and timing can know the foods really required for improvement degree of his or her symptoms as the appropriate number of candidates. In particular, regarding the number of candidates, even if the number of input symptoms is small, the user can know the enough number of candidates. On the other hand, even if the number of input symptoms is large, the user can also know a small number of candidates that contribute simultaneously for improving multiple symptoms.

(2) The user can know required foods after reflecting his or her preference on the score of the foods. Further, the user can record appropriately the proposed foods, and the foods selected by the user himself or herself from among the proposed foods.

(3) The user can know the required foods that have been selected from among multiple food groups.

(4) The user can input his or her symptom in a format of answering to the understandable questions to easily recognize the required foods and the foods selected by the user himself or herself.

(5) The user can review the nutrient that is effective for his or her physical constitution.

(6) The user doesn't need to answer repeatedly to the question which may lead to the same answer.

(7) The user can know the required foods with high accuracy by quantitative calculation using vectors.

The present invention is not limited to the above-described embodiment but includes various modifications. For example, the above-described embodiments are explained in details for better understanding of the present invention and are not limited to those including all the configurations described above. A part of the configuration of one embodiment may be replaced with that of another embodiment; the configuration of one embodiment may be incorporated to the configuration of another embodiment. A part of the configuration of each embodiment may be added, deleted, or replaced by that of a different configuration.

The above-described configurations, functions, processing modules, and processing means, for all or a part of them, may be implemented by hardware, for example, by designing an integrated circuit, and may be implemented by software, which means that a processing part interprets and executes programs providing the functions. The information of programs, tables, and files to implement the functions may be stored in a storage device such as a memory, a hard disk drive, or an SSD (Solid State Drive), or a storage medium such as an IC card, an SD card, or a DVD.

The drawings illustrate control lines and information lines as considered necessary for explanation but do not illustrate all control lines or information lines in the products. It can be considered that almost of all components are actually interconnected.

### REFERENCE SIGNS LIST

1 Food proposing system
2 Terminal
3 Network
4 External system
11 Central controller
12 Main storage
13 Auxiliary storage
14 Communication device
15 Input device
16 Output device
21 Symptom receiving and processing section
22 Required nutrient determining section
23 Food candidate determining section
24 Food candidate indicating and processing section
31 Symptom reception information
32 Food proposing information
33 Symptom and nutrient information
34 Food and nutrient information
51 Interview screen
61 Food proposing screen
71 Achievement rate transition screen
81 Conversation screen

What is claimed is:

1. A food proposing system comprising:
a storage section storing:
symptom and nutrient information including symptoms, nutrients, an effect degree of each of the nutrients for improving each of the symptoms, for each combination of the symptoms and the nutrients and
food and nutrient information including foods, the nutrients, a content of each of the nutrients contained in each of the foods, for each combination of the foods and the nutrients;
a symptom receiving and processing section configured to receive symptoms input by a user;
a required nutrient determining section configured to:
search the symptom and nutrient information on the input symptoms and
retrieve the effect degree of each of the nutrients for improving the input symptoms;
a food candidate determining section configured to:
compute a score of each of the foods on the basis of the retrieved effect degree of the each of the nutrients and the content thereof in the each of the foods included by the food and nutrient information, and
determine a food by an arbitrarily preset number, the food to be proposed to the user, based on how large computed scores are; and
a food candidate indicating and processing section configured to indicate the determined food.

2. The food proposing system according to claim 1, wherein
the food and nutrient information
includes a preference coefficient in association with each of the foods,
the food candidate indicating and processing section
receives a food selected by the user from the indicated foods, and
the food candidate determining section
computes the score on the basis of the preference coefficient and
updates the preference coefficient on the basis of a ratio of the selected food to the indicated foods.

3. The food proposing system according to claim 2, wherein
the food and nutrient information
includes food groups to which foods belong in association with the foods,
the food candidate determining section
determines a food to be proposed to the user from among each of the food groups.

4. The food proposing system according to claim 3, wherein
the symptom receiving and processing section
displays a question to ask either presence or absence of the symptom to a terminal operated by the user, and
the food candidate indicating and processing section
indicates the determined food to the terminal, as an icon, and
indicates the icon of the determined food selected by the user in such a manner as to be distinguished from other icons.

5. The food proposing system according to claim 4, wherein
the required nutrient determining section
updates the effect degree on the basis of an improvement degree for the each of the symptoms and a number of times of selection of the indicated foods.

6. The food proposing system according to claim 5, wherein
the symptom and nutrient information includes a question period in association with the each of the symptoms,
the symptom receiving and processing section displays the question to ask either presence or absence of the each of the symptoms on the basis of the question period.

7. The food proposing system according to claim 1, wherein
the symptom and nutrient information includes a first vector having components of the effect degrees of the nutrients for the each of the symptoms,
the food and nutrient information includes a second vector having components of the contents of the nutrients for the each of the foods, and
the food candidate determining section computes an inner product of the first vector and the second vector to compute the score.

8. A food proposing method, comprising steps of:
storing, by a storage section of a food proposing system, symptom and nutrient information including symptoms, nutrients, an effect degree of each of the nutrients for improving each of the symptoms, for each combination of the symptoms and the nutrients and
food and nutrient information including foods, the nutrients, a content of each of the nutrients contained in each of the foods, for each combination of the foods and the nutrients;
receiving, by a symptom receiving and processing section of the food proposing system,
symptoms input by a user;
searching, by an required nutrient determining section of the food proposing system,
the symptom and nutrient information on the input symptoms and
retrieving, by an required nutrient determining section of the food proposing system,
the effect degree of each of the nutrients for improving the input symptoms;
computing, by a food candidate determining section of the food proposing system, a score of each of the foods on the basis of the retrieved effect degree of the each of the nutrients and the content thereof in the each of the foods included by the food and nutrient information and
determining, by a food candidate determining section of the food proposing system,
a food by an arbitrarily preset number, the food to be proposed to the user, based on how large computed scores are; and
indicating, by a food candidate indicating and processing section,
the determined food on a display unit.

9. A food proposing program for implementing a food proposing system, the program causing a computer to carry out the steps of:
storing, by a storage section of a food proposing system, symptom and nutrient information including symptoms, nutrients, an effect degree of each of the nutrients for improving each of the symptoms, for each combination of the symptoms and the nutrients and
food and nutrient information including foods, the nutrients, a content of each of the nutrients contained in each of the foods, for each combination of the foods and the nutrients;
receiving, by a symptom receiving and processing section of the food proposing system,
symptoms input by a user;
searching, by an required nutrient determining section of the food proposing system,
the symptom and nutrient information on the input symptoms and
retrieving, by an required nutrient determining section of the food proposing system,
the effect degree of each of the nutrients for improving the input symptoms;
computing, by a food candidate determining section of the food proposing system, a score of each of the foods on the basis of the retrieved effect degree of the each of the nutrients and the content thereof in the each of the foods included by the food and nutrient information and
determining, by a food candidate determining section of the food proposing system,
a food by an arbitrarily preset number, the food to be proposed to the user, based on how large computed scores are; and
indicating, by a food candidate indicating and processing section,
the determined food on a display unit.
